(19) United States
(12) Patent Application Publication
(10) Pub. No.: US 2007/0286878 A1
(43) Pub. Date: Dec. 13, 2007

(54) REMOVABLE FILMS FOR SANITIZING SUBSTRATES AND METHODS OF USE THEREOF

(76) Inventor: Issifu I. Harruna, Conyers, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 11/422,715

(22) Filed: Jun. 7, 2006

(51) Int. Cl. A61K 9/70; A01N 25/00

(52) U.S. Cl. 424/405; 424/443

(57) ABSTRACT

Described herein are removable films for sanitizing substrates and methods of use thereof.

### BACKGROUND

It is well understood that microbiological pathogens are the primary cause of infectious diseases in humans. For example, eighty percent of all infections ranging from the usually benign cold and more debilitating flu to the truly horrific deadly Ebola are transmitted by direct contact. The Centers for Disease Control and Prevention estimate more than one-third of healthcare-associated infections can be prevented through better infection control programs such as cleaning exposed surfaces. The reasons most often cited for failing to clean surfaces adequately are inconvenience and lack of time.

In March 2001, an article in Emerging Infectious Diseases entitled “Antiseptic Technology: Access, Affordability, and Acceptance” further reinforces the findings that time and convenience are critical compliance factors. In the October 2000 issue of Family Medicine (“Alcohol-free Instant Hand Sanitizer Reduces Elementary School Illness Absenteeism”), a remarkable reduction in absenteeism was observed when sanitizers were introduced in public school classrooms. Results showed students using sanitizers were found to have 41.9% fewer illness-related absence days, representing a 28.9% and a 49.7% drop in gastrointestinal- and respiratory-related illness, respectively. In summary, daily use of a sanitizer was associated with significantly lower rates of illness-related absenteeism.

Therefore, what is currently lacking is a sanitizing device that permits wide distribution, ready access, convenience, inconspicuous, and omnipresent. The state of the art does not provide either devices or methods that adequately respond to these requirements. Dispensers hung on walls or set on counters have proven only marginally effective in even the controlled environments of hospitals and schools. One approach as described herein involves the development and application of an inexpensive, disposable, multi-dose, convenient, ubiquitous, inconspicuous, sanitizing device that can be accessed in a timely manner and reused several times during the course of a day's normal activities. Successful methods promoting the wide distribution of such a device would contribute to eventual habitual use.

### SUMMARY

Described herein are removable films for sanitizing substrates and methods of use thereof. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. Like numbers represent the same elements throughout the figures.

FIG. 1 shows an exemplary device as applied to a substrate.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Described herein are devices for sanitizing substrates. In one aspect, the device comprises a base film and a plurality of thin films, wherein the base film and each thin film have a first surface and a second surface, wherein the second surface of the base film is attached to the first surface of a thin film, wherein the first surface of each thin film is attached to the second surface of each previous thin film, wherein the base film and each thin film comprises at least one antimicrobial agent, wherein the antimicrobial agent is not photosensitive, and wherein the base film and each thin film are removable.

In another aspect described herein is a device for sanitizing a substrate, comprising a base film and a plurality of thin films, wherein the base film and each thin film have a first surface and a second surface, wherein the second surface of the base film is attached to the first surface a thin film, wherein the first surface of each thin film is attached to the second surface of each previous thin film, wherein each thin film comprises at least one antimicrobial agent incorporated in the thin film, and wherein the base film and each thin film are removable.

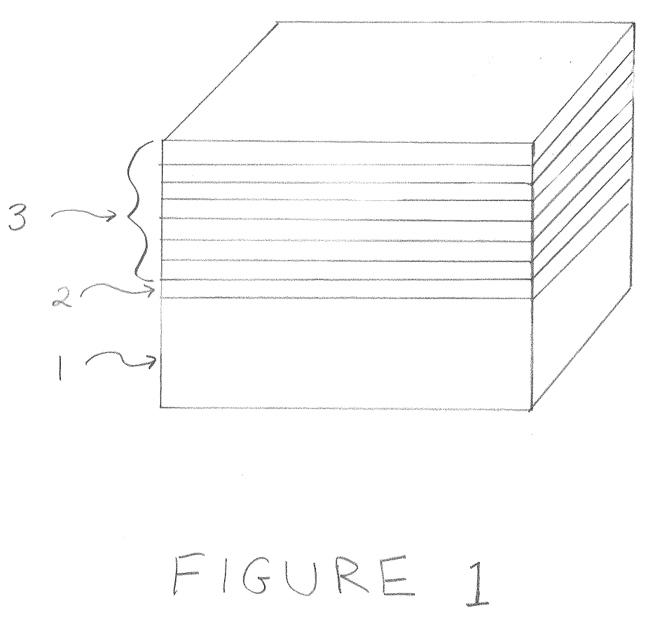

FIG. 1 depicts one aspect of the device described herein as applied to a substrate. The device is composed of base film 2 and a plurality of thin films (3). The base film is adjacent to substrate 1. In FIG. 1, the base layer (i.e., the first surface of the base film) is in intimate contact with the surface of the substrate; however, it is contemplated that one or more intermediate layers can be between the base film and the substrate. Modes of attaching the base film to the substrate will be discussed below. Next, one or more thin films can be attached to the second surface of the base film. The number of thin films that can be applied to the base film can vary depending upon the substrate that the device is applied to. Modes of attaching the thin films to the base film as well as to one another will be discussed below.

A variety of materials can be used for the base and thin film. The selection of the material can vary depending upon the end-use of the device. Factors to consider in selecting the material for the films include, but are not limited to, flexibility, hydrophobicity, and degradability. In one aspect, the material is a non-woven fabric or paper.

In another aspect, the films are composed of one or more polymers. A variety of polymers can be used to produce the base and thin films. It is possible to use different polymeric materials to produce the base and thin films; however, in certain aspects, the base film and each thin film are composed of the same polymer. Examples of polymers useful for producing the base and thin films include, but are not limited to, low-density polyethylene, high-density polyethylene, low-density polyethylene, a polystyrene, a polyester, a propylene homopolymer, an ethylene-propylene random copolymer, an ethylene-propylene block copolymer, an ethylene-propylene-butene terpolymer, an ethylene-vinyl acetate copolymer, an ethylene-unsaturated carboxylic acid ester copolymer, an ethylene-unsaturated carboxylic acid metal salt copolymer, a polyvinyl alcohol, polyvinylidene chloride, a polyamide, a polyacrylonitrile, a propylene-vinyl chloride copolymer, a propylene-butene copolymer, a propylene-maleic anhydride copolymer, a propylene-olefin copolymer, an unsaturated carboxylic acid-modified polyethylene, an unsaturated carboxylic acid-modified polypropylene, an ethylene-propylene rubber, an atactic polypropylene, a polyimide, a polyvinylpyrrolidone, a polyacrylamide, a polystyrenesulfonic acid, a polyimine, a polyquaternary ammonium salt, a modified starch, a modified cellulose, a polyvinylpyridine, a polyvinylacetate, a polyvinylether, a polyvinyloxazolidone, a polyhaloalkylene, a polyurethane, a polysilane, a polysiloxane, a polyceramic, a polyglutamate, or a combination thereof.

In one aspect, the base film and the thin film comprise a low density polyethylene (LDPE), linear low density polyethylene (LLDPE), high density polyethylene (HDPE), polypropylene, ethylene vinyl acetate copolymer (EVA), ethylene-(meth)acrylic acid copolymer (EAA or EMA), poly(hexamethylene sebacamide) (nylon 6,10), poly(hexamethylene adipamide) (nylon 6,6), poly epsilon caprolactam (nylon-6), poly(ethylene terephthalate), or a combination thereof.

The base film and the thin films are attached to one another by various techniques. In one aspect, the base film and each thin film are attached to one another by electrostatic forces. For example, the second surface of the base film can be prepared or modified such that it has a slight positive or negative charge. If the first surface of the thin film has the opposite charge at the surface, then the first surface of the thin film will be electrostatically attracted (i.e., attached) to the second surface of the base film.

In another aspect, the base film and thin films are attached to one another by an adhesive. In this aspect, the adhesive is applied to the first surface of the thin film using techniques known in the art. In one aspect, the adhesive can be applied to the first surface of each thin film followed by stacking of the thin films. Alternatively, the adhesive can be applied in such a manner that a molten adhesive is extruded during extrusion of the thin/base film. The selection of the adhesive can vary depending upon the composition of the base/thin films and the substrate that the device is applied to. The adhesive will permit easy removal of each thin film and base film but still provide good attachment to the substrate so that the films do not rub off upon contact. With respect to the base film, an adhesive that is the same or different from that used to attach the thin films can be used to attach the device (i.e., the first surface of the base film) to the substrate. Examples of adhesives useful herein include, but are not limited to, an ethylene-vinyl acetate copolymer, an ethylene-methyl acrylate copolymer, a mixture of an ethylene-vinyl acetate copolymer and an ethylene-methyl acrylate copolymer, a styrene block-butadiene block copolymer elastomer, a styrene-butadiene copolymer elastomer, or a combination thereof. In another aspect, the polyvinylpyrrolidone acrylic acid polymers disclosed in U.S. Pat. No. 5,645,855, which are incorporated by reference, can be used as the adhesives.

The base film and the thin films comprise at least one antimicrobial agent. It is also contemplated that when an adhesive is used, the adhesive can contain one or more antimicrobial agents. The term “antimicrobial agent” as used herein is defined as a substance that can retard, reduce, inhibit, or eliminate microbial growth.

In one aspect, the antimicrobial agent comprises a percarboxylic acid, a halogen composition or interhalogen thereof, a halogen donor composition, chlorine dioxide, ozone, a quaternary ammonium compound, an acid-anionic organic sulfonate or sulfate, a protonated carboxylic acid, a metal ion, or a combination thereof. Non-limiting examples of percarboxylic acids include: C₁-C₁₀ percarboxylic acids, diperoxyglutaric acid, diperoxyadipic acid, diperoxysuccinic acid, diperoxysuberic acid, diperoxymalonic acid, peroxylactic acid, peroxyglycolic acid, peroxyoxalic acid, peroxypyruvic acid, and mixtures thereof. Non-limiting examples of halogen compounds and interhalogens thereof include: Cl₂, Br₂, I₂, ICl, IBr, ClBr, ICl₂⁻, IBr₂⁻, and mixtures thereof. Non-limiting examples of halogen donor compositions include: HOCl, HOI, HOBr, and the salts thereof, chlorhexidine, N-iodo, N-bromo, or N-chloro compounds, N-bromosuccinamide, chloroisocyanuric acid, or 2-N-sodium-N-chloro-p-toluenesulfonamide. A non-limiting example of chlorine dioxide compositions includes chlorine dioxide generated from conventional chemical generators such as those sold by Prominent™ or preferably generated electrochemically using Halox™ generators. A non-limiting example of ozone includes ozone-generated electrochemically via high voltage discharge in oxygen. Non-limiting examples of quaternary ammonium compounds include: didecyldimethylammonium chloride, dioctyldimethylammonium chloride, octyldecyldimethylammonium chloride, alkyldimethylbenzylammonium chloride, and mixtures thereof. Non-limiting examples of acid-anionic organic sulfonates and sulfates include: acidic solutions of linear benzylsulfonic acid and sulfonated oleic acid. Non-limiting examples of protonated carboxylic acids include: solutions with a pH less than 5 of one or more C₁-C₁₀carboxylic acids. Non-limiting examples of metal ions include ions of Ag, Au, Pt, Pd, Os, Ir, Ru, Rh, (i.e. the noble metals), Cu, Sn, Sb, Bi and Zn. In other aspects, the antimicrobial agent also includes nanoparticles with anti-pathogenic activity. U.S. Pat. Nos. 4,051,058; 4,051,059; 5,200,189; 5,200,198; 5,489,434; 5,718,910; 5,314,687; and 5,437,868 provide further information with respect to the formation of antimicrobial agent formulations. These patents are incorporated herein by reference in their entirety.

In certain aspects, the antimicrobial agent is not photosensitive. The term “photosensitive” as used herein is defined as any compound that is inactive until it is exposed to light to become active (i.e., possess antimicrobial activity). For example, the photosensitizer can produce singlet oxygen upon illumination with light, where the singlet oxygen can behave as an antimicrobial agent.

The base film and thin film can include other components besides the antimicrobial agent, including a deodorizing agent, a perfume, or a combination thereof.

Deodorant and deodorizer are defined here as any product or products designed or used to reduce or eliminate offensive odors. Depending upon the deodorant selected, the deodorant can remove odorous molecules by a variety of mechanisms (e.g., absorption). A variety of different deodorants known in the art can be used herein.

Many types of perfumes can be employed, and their selection is based upon their compatibility with the films and antimicrobial agents. Suitable perfumes include, but are not limited to, fruits such as almond, apple, cherry, grape, pear, pineapple, orange, strawberry, raspberry; musk, and flower scents such as lavender-like, rose-like, iris-like, and carnation-like. Other pleasant scents include herbal and woodland scents derived from pine, spruce, and other forest smells. Perfumes can also be derived from various oils, such as essential oils, or from plant materials such as peppermint, spearmint and the like. A list of suitable perfumes is provided in U.S. Pat. No. 4,534,891, the contents of which are incorporated by reference in its entirety. Another source of suitable perfumes is found in Perfumes, Cosmetics and Soaps, Second Edition, edited by W. A. Poucher, 1959. Among the perfumes provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like. U.S. Pat. Nos. 2,947,780 and 6,632,788, among various other patents, disclose cyclic chemicals that are suitable for use as fragrance chemicals. Those with skill in the art appreciate how differences in the chemical structure of the molecule can result in significant differences in the odor, notes and characteristics of a molecule.

The antimicrobial agent and other optional components can be applied to the base and thin films using techniques known in the art. In one aspect, the antimicrobial agent is incorporated in the base film and each thin film. For example, the antimicrobial agent can be blended with one or more polymers then extruded to produce a film (base or thin film). In this aspect, the antimicrobial agent is dispersed throughout the entire film. In other aspects, the antimicrobial and optional components can be coated to the surface of the films. Here, the antimicrobial agent is at the surface of the film or just below the surface. The antimicrobial agent and optional components can be coated on the films using techniques known in the art, including smearing, grafting, or spraying the components on the films or dipping the films in solutions of antimicrobial agent. The amount of antimicrobial agent present in the films can vary depending upon the selection of the antimicrobial agent, the base/thin films, and the end-use of the device.

In certain aspects, it may be desirable for the base film and each thin film to be printable or writable. For example, it is contemplated that various types of information such as instructional information, warnings, tradename and/or trademark, logo, copyright symbol, other symbols, advertising information, and the like can be printed on the base film or thin film. It is contemplated that printed information can be on either side of the films.

The devices herein can be used to sanitize a substrate (i.e., prevent microorganisms from being passed to a subject upon contact with the substrate). In one aspect, described herein is a method for protecting a substrate, comprising attaching the first surface of the base film of any device described herein to the exposed surface of the substrate. The devices described herein can be readily attached to a substrate by attaching the first surface of the base film to the exposed surface of the substrate using techniques described herein. In one aspect, a protective tape or paper can be peeled from the first surface of the base layer, exposing an adhesive that can readily stick to the exposed surface of the substrate.

Once the device is attached to the substrate, the antimicrobial agent(s) present on the films can prevent the passage of microorganisms to a subject. After a certain period of time, the effectiveness of the antimicrobial agent is diminished, at which time the thin film is removed to expose a second thin film. The thin films can be readily removed without tearing or damaging the underlying thin film. In order to facilitate the removal of the thin film, a tab may be attached to each film (base and thin).

A variety of different substrates can be sanitized using the devices described herein. The substrates can be shielded regardless of their form, shape, or orientation so long as they are solid. Examples of substrates that can be sanitized using the devices described herein are as follows:

1. In dentistry, apparent surface areas of concern are found on the chair adjustment knob, the instrument tray, instrument supply drawer handles, water faucets, examination light adjustment handle, timers, mixers, etc. i.e. anything that the dentist or assistant may wish to access during the course of the procedure. These items apply equally to dental hygienist operational procedures as to the dentist's procedures.

2. In a medical doctor's office, examination table adjustment surfaces, light switches, cabinet access knobs, chairs, examination lights, etc., i.e., anything which the doctor or assisting operative may need to contact during a given procedure that he/she may have contacted during a previous procedure or may need to contact again in a subsequent procedure.

3. In all human surgical operations, there are many objects that should be sanitized. For example, surgical operation lighting and examination lamps, rails on gurneys, adjustment levers on operation tables, various patient positioning appliances and hardware, control switches and adjustment knobs located on a great variety of life support systems such as dialysis machines, heart lung machines, vital sign monitoring equipment, electrical shock paddles, etc.

4. In the medical, research, or pathology lab, typical examples of items that can be sanitized are microscopes, autoclaves, centrifuges, test tube racks, incubation chambers, etc.

5. In health care applications such as nursing homes typical examples are bedpans, flushing controls, toilet seats, blood sampling and handling apparatus, health care equipment controls, etc.

6. In the food service industry, for example in a serving line where a food server wearing food laden latex gloves may have need to adjust warning lamps, or open refrigerator doors, etc. where it would suggest good sanitation practice to not leave deposits of grease or food bits on surfaces to putrefy before subsequent days or weeks serving activities.

7. Similarly in the residential home, sanitizing various surfaces such as sink faucets, refrigerator handles, dishwasher handles, toilets, countertops, phone handsets, computer keyboards, etc. Also contemplated are baby changing stations, which can be residential dwellings as well as in public restrooms.

8. The devices described herein can be used in a variety of different articles of clothing. For example, the device can be applied to the insole of a shoe.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A device for sanitizing a substrate, comprising a base film and a plurality of thin films, wherein the base film and each thin film have a first surface and a second surface, wherein the second surface of the base film is attached to the first surface of a thin film, wherein the first surface of each thin film is attached to the second surface of each previous thin film, wherein the base film and each thin film comprises at least one antimicrobial agent, wherein the antimicrobial agent is not photosensitive, and wherein the base film and each thin film are removable.

2. The device of claim 1, wherein the base film and each thin film comprise the same material.

3. The device of claim 1, wherein the base film and each thin film comprise a non-woven fabric or paper.

4. The device of claim 1, wherein the base film and each thin film comprise a polymer.

5. The device of claim 4, wherein the polymer comprises a low-density polyethylene, a high-density polyethylene, a low-density polyethylene, a polystyrene, a polyester, a propylene homopolymer, an ethylene-propylene random copolymer, an ethylene-propylene block copolymer, an ethylene-propylene-butene terpolymer, an ethylene-vinyl acetate copolymer, an ethylene-unsaturated carboxylic acid ester copolymer, an ethylene-unsaturated carboxylic acid metal salt copolymer, a polyvinyl alcohol, polyvinylidene chloride, a polyamide, a polyacrylonitrile, a propylene-vinyl chloride copolymer, a propylene-butene copolymer, a propylene-maleic anhydride copolymer, a propylene-olefin copolymer, an unsaturated carboxylic acid-modified polyethylene, an unsaturated carboxylic acid-modified polypropylene, an ethylene-propylene rubber, an atactic polypropylene, a polyimide, a polyvinylpyrrolidone, a polyacrylamide, a polystyrenesulfonic acid, a polyimine, a polyquaternary ammonium salt, a modified starch, a modified cellulose, a polyvinylpyridine, a polyvinylacetate, a polyvinylether, a polyvinyloxazolidone, a polyhaloalkylene, a polyurethane, a polysilane, a polysiloxane, a polyceramic, a polyglutamate, or a combination thereof.

6. The device of claim 4, wherein the polymer comprises a low density polyethylene (LDPE), linear low density polyethylene (LLDPE), high density polyethylene (HDPE), polypropylene, ethylene vinyl acetate copolymer (EVA), ethylene-(meth)acrylic acid copolymer (EAA or EMA), poly(hexamethylene sebacamide) (nylon 6,10), poly(hexamethylene adipamide) (nylon 6,6), poly epsilon caprolactam (nylon-6), poly(ethylene terephthalate), or a combination thereof.

7. The device of claim 1, wherein the base film and each thin film are attached to one another by an adhesive.

8. The device of claim 7, wherein the adhesive comprises an ethylene-vinyl acetate copolymer, an ethylene-methyl acrylate copolymer, a mixture of an ethylene-vinyl acetate copolymer and an ethylene-methyl acrylate copolymer, a styrene block-butadiene block copolymer elastomer, a styrene-butadiene copolymer elastomer, or a combination thereof.

9. The device of claim 1, wherein the base film and each thin film are attached to one another by electrostatic forces.

10. The device of claim 1, wherein the antimicrobial agent comprises a percarboxylic acid, a halogen composition or interhalogen thereof, a halogen donor composition, chlorine dioxide, ozone, a quaternary ammonium compound, an acid-anionic organic sulfonate or sulfate, a protonated carboxylic acid, a metal ion, or a combination thereof.

11. The device of claim 1, wherein the antimicrobial agent is incorporated in the base film and each thin film.

12. The device of claim 1, wherein the antimicrobial agent is coated on the base film and each thin film.

13. The device of claim 1, wherein the base film and each thin film further comprise a deodorizing agent, a perfume, or a combination thereof.

14. The device of claim 1, wherein the base film and each thin film comprise a tab attached to each film.

15. The device of claim 1, wherein the first surface or second surface of the base film and each thin film is writable.

16. A device for sanitizing a substrate, comprising a base film and a plurality of thin films, wherein the base film and each thin film have a first surface and a second surface, wherein the second surface of the base film is attached to the first surface a thin film, wherein the first surface of each thin film is attached to the second surface of each previous thin film, wherein each thin film comprises at least one antimicrobial agent incorporated in the thin film, and wherein the base film and each thin film are removable.

17. A method for sanitizing a substrate, comprising attaching the first surface of the base film of the device of claim 1 to the exposed surface of the substrate.

18. The method of claim 17, wherein the substrate comprises a tabletop, a toilet seat, or a baby changing station.

19. A method for sanitizing a substrate, comprising attaching the first surface of the base film of the device of claim 16 to the exposed surface of the substrate.

20. The method of claim 19, wherein the substrate comprises a tabletop, a toilet seat, or a baby changing station.
